(19) United States
(12) Patent Application Publication
(10) Pub. No.: US 2012/0316521 A1
(43) Pub. Date: Dec. 13, 2012

(54) Manual negative pressure dressing-Webster's Dressing

(76) Inventor: Ronaldo Scholze Webster, (US)

(21) Appl. No.: 13/171,140

(22) Filed: Jun. 28, 2011

Related U.S. Application Data
(60) Provisional application No. 61/495,227, filed on Jun. 9, 2011.

(51) Int. Cl. A61M 1/00

(52) U.S. Cl. 604/319

(57) ABSTRACT

Vacuum therapy is an effective way to treat complex open wounds. Today, most of the equipment is very expensive to provide widespread use of this therapy and, most of them, are designed to in-patient use. Also, there are difficulties to obtain the needed materials in the same package for specific use of treating open wounds, which causes delay in the treatment. We designed a kit that is cost-effective and ready-to-use to be used with Jackson-Pratt or Portovac style suction drains that allows a cheap, out-patient alternative for use in open-wound situations. We devise that this system could be used in emergency situations where there is a hostile environment such as natural catastrophes, war and emergency situations, as well as to regular in and out-patient wound care.

### CROSS-REFERENCE TO RELATED APPLICATIONS

Application No. 61/495,227 (provisional)

The USPTO has received this submission at 17:54:31 Eastern Time on Jun. 9, 2011.

### STATEMENT REGARDING FEDERALLY SPONSORED RESEARCH OR DEVELOPMENT

This model of utility wasn't made under any federally sponsored research and development.

### BACKGROUND OF THE INVENTION

The device planned is to be used in human complex open wounds due to traumatic, neoplastic or post-surgical origins and is devised to be used in hostile environment and emergency situations, as well as, in and out-patient wound care.

The purpose of this patent is explore a cost-effective way to provide vacuum-assisted wound closure using a manual pump device (Jackson-Pratt or Portovac Style, specifically) to achieve negative pressure. This alternative vacuum device can help greater numbers of subjects than is the case today, with results similar to those of more expensive methods.

This treatment still investigational this time but case reports and series of cases were already done with seriously encouraging results. Since 2005, according to prior unpublished results of the author, and a case report of a 17 y.o. child, followed from 2009 to 2011 (which was awarded by the Brazilian Society of Plastic Surgery in May, 2011 and published in the Plastic and Reconstructive Surgery Journal under the ID number PRS-S-11-01343-1), it is our belief that this treatment should be highly considered. Further clinical research looking forward widespread use of this system certainly will be conducted in the near future to better clarify the actual outcomes of this dressing.

The claimed dressing kit is handcrafted using polyurethane or polivynil-alcohol foam (preferably an open cell kind of foam). The manual pump tube is inserted into the foam and the components are placed on the wound over a of non-adherent gauze. An outer auto-adhesive plastic sheet layer prevents air leakage after activating negative pressure using the manual pump (specifically a Jackson-Pratt or Portovac type). The device needs to be rechecked periodically for fluids and proper vacuum maintenance, and the dressing needs to be changed completely according medical advice.

Historic Background:

General vacuum treatment was developed from a 2000 TEC Assessment (1) that evaluated negative pressure therapy of pressure ulcers, venous ulcers, and diabetic ulcers. [0009] The Assessment offered the following observations and conclusions:
One randomized controlled trial (RCT) was identified, which included 24 patients with a total of 36 wounds, mostly pressure ulcers. The study reported statistically significant improvements in some parameters of wound healing. However, there were significant methodologic flaws in the study. For example, partial wound closure was used as the primary outcome of interest instead of the more clinically relevant complete closure. In addition, as wounds vary pathophysiologically, stratification of wound size, duration, and location is necessary. All of these variables were reported in aggregate form, making the finding impossible to interpret.
Uncontrolled trials, frequently using patients as their own controls, have reported favorable results. However, due to the multimodality nature of wound care, randomized trials are necessary to isolate the individual contribution of negative pressure therapy to overall wound care.
The efficacy of negative pressure therapy compared to standard wound management should be determined by high-quality clinical trials that contain the following features:
enrollment of a patient population with ulcers refractory to standard treatment after an appropriate period of optimal wound management;
randomized assignment to treatment group;
treatment in the control arm that includes all of the main components of optimal wound care, e.g., debridement, irrigation, whirlpool treatments, and wet-to-dry dressings;
outcome measure(s) that represent clinically important end points, such as the percent of patients with complete healing or the percent of patients that require skin grafting.

Policy updates thereafter focused on comparative trials with the features described in the TEC Assessment conclusions, e.g., enrollment of patients with ulcers refractory to standard treatment, randomization, optimal standard wound care treatment in the control arm, and clinically important end points. Published trials have continued to have small numbers of subjects as well as methodological weaknesses and have failed to meet one or more of the criteria suggested by the TEC Assessment.

One trial by Ford and colleagues (2) compared negative pressure therapy with an alternative wound healing system (HealthPoint system, consisting of 3 FDA-approved gel products: Accuzyme, Iodosorb, and Panafil) and did not included a standard wound care treatment group. Thus, this trial did not provide any data on the efficacy of negative pressure therapy compared with standard wound care.

Another was a small trial that randomized 24 patients with pressure ulcers of the pelvic region to negative pressure therapy or standard wound care. (3) All patients with pelvic pressure sores were eligible for enrollment and were not required to be refractory to standard treatment. Patients in the control group received wet-to-dry dressings, but may not have received other aspects of optimal wound management such as debridement or whirlpool therapy. Finally, the main outcome measure, 50% reduction in wound size, is not necessarily a clinically important outcome when compared with other potential outcomes such as complete wound healing. There were no significant group differences for the main outcome measure, time to 50% reduction of wound volume (27±10 days in the negative pressure therapy group and 28±7 days in the control group). However, these results are subject to bias, given the small number of patients in the study and the lack of power calculations.

A trial by Doss et al. was nonrandomized and featured a non-concurrent control group. (4) These authors performed a retrospective comparison of negative pressure therapy with conventional wound management for patients with post-sternotomy osteomyelitis. Treatment assignment was at the discretion of the treating surgeon and was mainly dependent on the time period during which the patient was treated. Treatment duration was shorter for the negative pressure therapy (17.2±5.8 vs. 22.9±10.8 days, p=0.009), as was length of hospital stay (27.2±6.5 vs. 33.0±11.0 days, p=0.03). [0014] Conclusions of this trial are limited by the potential for non-comparability of treatment groups due to non-randomized treatment assignment and the non-concurrent time period during which the control group was enrolled.

Eginton et al. (5) included patients with diabetic foot wounds that were not expected to heal within 1 month and reported on change in wound dimensions. Using a crossover design in 6 patients who first had 2 weeks of either moist dressings or negative pressure therapy, then switched to the other for 2 weeks, the mean change in area was an increase of 5.9% for the control intervention and a decrease of 16.4% for negative pressure therapy. The mean percent reduction in wound volume was 0.1% in the moist dressing phase and 59% in the negative pressure therapy phase (p 0.05); +2.4 and −12.9 (width, p>0.05); and −7.7 and −49 (depth, p

Moues et al. (6) reported on the time to readiness for surgical closure, among patients with full-thickness wounds of various etiologies. Log-rank test analysis of Kaplan-Meier time to readiness did not show any statistically significant differences between groups. The median time to readiness for surgical closure was 6 days for negative pressure therapy patients and 7 days for conventionally treated patients (p=0.19). The authors also found a significantly higher daily percent change in wound area among negative pressure therapy patients (3.8), compared with conventionally treated patients (1.7, p A 2004 systematic review published by the Agency for Healthcare Research and Quality concluded that available published trials “did not find a significant advantage for the intervention on the primary endpoint, complete healing, and did not consistently find significant differences on secondary endpoints and may have been insufficiently powered to detect differences”. (7)

Armstrong and colleagues (8) performed an RCT of NPWT using the V.A.C.® system (n=77) and standard moist wound care (n=85) to treat partial foot amputation wounds (average wound duration 1.5 months) in diabetic patients. Forty-three (56%) of 77 NPWT patients achieved complete closure during the 16-week assessment period versus 33 (39%) of 85 controls (p=0.040). Log-rank analysis showed the rate of complete closure was significantly faster with NPWT than in controls (p=0.005). Most wounds healed by secondary intention (NPWT, 31 [40%]; controls, 25 [30%]), with no significant difference between groups in the need for surgical closure (NPWT, 12 [16%]; controls, 8 [9%]). The frequency and severity of adverse events, most commonly infection (32% in both groups), were similar. Intention to treat analysis was reported, but substantial unaccounted loss to follow-up (23%), lack of allocation concealment in randomization, and between group differences in wound care limits these results. The authors reported a reanalysis of these data to examine the possible role of wound chronicity on healing in a later paper. (9) This analysis revealed no significant difference in the proportion of acute and chronic wounds that achieved complete wound closure with either therapy, although the Kaplan-Meier curve demonstrated statistically faster (p=0.03) healing in the NPWT group in both acute and chronic wounds. Further, in both the acute and chronic wound groups, results for patients treated with NPWT appeared superior to those in patients who received standard therapy. While these findings suggest that NPWT improves outcomes compared to standard care, this was a post-hoc, unplanned reanalysis of data from a study with several flaws and potential biases that limit validity.

Braakenburg and colleagues (10) compared NPWT using the V.A.C.® system (n=32) with conventional moist wound therapy (n=33) in patients with different types of wounds (operation wounds, diabetic ulcers, pressure sores) that were defined as acute (less than 48 hours old), subacute (less than 6 weeks), or chronic (longer than 6 weeks). The primary outcome, wound-healing time, was calculated from the date of initial debridement at study entry to the date of reaching an endpoint, defined as a complete granulated wound, or a wound ready for skin grafting or healing by secondary intention. Twenty-six (81%) NPWT patients and 19 (58%) conventional therapy patients reached an endpoint (chi-square=4.27, p<0.05). The median healing time was 4 days shorter in the NPWT group (16 days) compared with controls (20 days), a non-significant difference. NPWT did not significantly reduce bacterial load, enhance wound granulation, or increase the rate of wound closure compared with conventional care. Complications such as bleeding, fistula, osteomyelitis or sepsis did not occur. Substantial, unaccounted loss to follow-up (NPWT, 19%; controls, 36%) and ill-defined wound characteristics confound the results.

Vuerstaek and colleagues (11) compared the efficacy of NPWT using the V.A.C.® system (n=30) with conventional care (n=30) in patients hospitalized with chronic venous, combined venous and arterial, or microangiopathic (arteriolosclerotic) leg ulcers (ABI greater than 0.60) of greater than 6 months' duration after failure of ambulatory (longer than 6 months) or surgical options in an outpatient clinic according to the Scottish Intercollegiate Guideline Network (SIGN). (16) Following complete debridement, patients were randomly allocated to NPWT or conventional moist wound care until granulation tissue covered 100% of the surface. Full-thickness punch skin grafts from the thigh were applied, followed by 4 days of NPWT or conventional care to assure complete graft adherence. Each group then received standard care with nonadhesive dressings and compression therapy until complete healing (primary outcome) occurred. The median time to complete healing was 29 days (95% CI: 25.5-32.5) with NPWT and 45 days (95% CI: 36.2-53.8) in the controls (p=0.0001). Ninety percent of the ulcers treated with NPWT healed within 43 days, compared with 48% in the control group. A slightly greater number of adverse events occurred in the NPWT group (12 total) than in the controls (7 total), primarily due to cutaneous damage secondary to therapy (7 vs. 2, respectively). Ulcer relapse occurred in 52% (n=10) of NPWT patients compared with 42% (n=10) of controls (p=0.47). A total of 11 patients (18%) dropped out before follow-up was complete, but intention-to-treat analysis accounted for this. These results suggest NPWT significantly hastened wound healing, but the use of skin autografts makes it difficult to discern the contribution of NPWT to the primary outcome.

2008-2010 Update

A MEDLINE search for the period October 2007 to January 2010 found a number of systematic reviews of the literature on vacuum-assisted, also called negative pressure, wound therapy (NPWT) and 4 randomized, controlled trials, as well as reports of case series. A 2008 update of a 2002 Cochrane review of negative pressure wound therapy for treatment of chronic wounds was published. (12) Five RCTs were reviewed in this second update for a total of 7 trials involving 205 participants. The 7 trials compared NPWT with 5 different comparator treatments; 4 trials compared NPWT with gauze soaked in either 0.9% saline or Ringer's solution. The other 3 trials compared NPWT with hydrocolloid gel plus gauze, a treatment package comprising papain-urea topical treatment, and cadexomer iodine or hydrocolloid, hydrogels, alginate and foam. The authors report that the data do not show that NPWT significantly increases the healing rate of chronic wounds compared with comparators and concluded that “trials comparing NPWT with alternative treatments for chronic wounds have methodological flaws and data do demonstrate a beneficial effect of NPWT on wound healing however more, better quality research is needed.”

A 2007 Cochrane review of the literature on NPWT for treatment of partial thickness burns found only one randomized, controlled trial that satisfied the inclusion criteria, and the methodological quality of the trial was poor. (13) The authors concluded that there is a “paucity of high quality [randomized, controlled trials] on NPWT for partial thickness burn injury with insufficient sample size and adequate power to detect differences, if there are any, between NPWT and conventional burn wound therapy dressings.” Gregor and colleagues included nonrandomized trials in their review if there was a concurrent control group, and concluded that though there is some indication that negative pressure wound therapy may improve wound healing, the evidence is insufficient to clearly prove an additional benefit. They note that the large number of prematurely terminated and unpublished trials of the therapy is reason for concern. (14) Authors of other systematic reviews, even if they conclude that there is evidence of efficacy, call for larger, high quality studies. (15, 16)

Of the randomized, controlled trials identified in the most-recent literature search, one compared negative pressure wound therapy with advanced moist wound therapy (predominantly hydrogels and alginates) and was not considered in this update. The second was a post hoc retrospective study of resource utilization and costs in a study reviewed in a previous update. (8) The third describes a randomized, controlled trial of NPWT carried out in India using a locally constructed device. (17) In this study, 48 patients were randomized to NPWT or moist dressings. Wound etiologies were diabetic foot ulcers (n=15), pressure ulcers (n=11), cellulitis/fasciitis (n=11), and “other” (n=11). One patient in the NPWT group and 12 in the conventionally treated group were lost to follow-up. No statistically significant differences in time to closure were observed between groups except in a subset analysis of pressure ulcers (mean 10 [±7.11] days for the treatment group and 27 [±10.6] days in controls [p=0.05]). NPWT-managed wounds in patients who remained hospitalized closed faster than gauze-dressed wounds. The high drop-out rate prevents drawing clear conclusions from this study.

Interest in the use of this modality for management of surgical and traumatic wounds is reflected in the large number of articles reporting on these applications in the recent literature. Three non-randomized comparative studies were identified in the literature search. (A preliminary report of 2 randomized, controlled trials evaluating NPWT to treat hematomas and surgical incisions following high-energy trauma was published in 2006, however no report of the completed study was found.) (18) Shilt et al. compared outcomes for 16 children treated with NPWT after lawnmower injuries to outcomes for 15 historic controls treated with wet-dry or Xeroform dressings. (19) There were no differences in infection rates between groups and patients treated with NPWT had longer hospital stays. Fifty-three percent of the controls required a free flap versus 19% of the NPWT group. The small number of subjects in this study limits interpretation of the results as does the lack of a comparable control group. Yang and colleagues retrospectively reviewed records of 34 patients who underwent NPWT after fasciotomy wounds for traumatic compartment syndrome of the leg and compared them with matched historic controls measuring time to definitive closure (delayed closure with sutures or skin graft). (20) Average time to definitive closure for both lateral and medial wounds was 6.7 days in the NPWT group (68 wounds in 34 patients) and 16.1 days in the controls (70 wounds in 34 patients) (p<0.05).

In another study of fasciotomy wounds, Zannis et al. retrospectively reviewed records of patients with upper and lower extremity fasciotomy wounds treated over a 10-year period. (21) Some wounds were treated exclusively with NPWT, some with only normal saline wet-to-dry dressings, and some with both according to surgeon preference. Of 142 upper extremity wounds, 74 received conventional treatment and 68 were treated with NPWT. Of 662 lower extremity wounds, 196 received only conventional treatment, 370 received only NPWT, and 96 received both treatments. The authors report a higher rate of primary closure using NPWT (82.7%) versus wet-to-dry dressings (p<0.05) for all lower extremity wounds (this appears to include wounds treated with the combination of NPWT and wet-to-dry dressings), and 55.6% (p<0.03) for upper extremity wounds. Lack of an equivalent contemporaneous control group limits the application of these findings.

A large number of case series are reported. Two papers describe case series of patients treated with NPWT after deep wound infections following spinal fusion. (22) A prospective study of 6 children reported that infection (all caused by Staphylococcus aureus, one of them methicillin resistant and one in which Enterobacter cloacae also cultured) was controlled in all children and removal of instrumentation was not required in any. Time to wound closure averaged 3 months. Ploumis et al. retrospectively reviewed 73 patients who had 79 wound infections after undergoing spine surgery. (23) Sixty patients had implants (instrumentation or allograft) within the site of wound infection, and 6 had recurrent infections. Once NPWT was initiated, there was an average of 1.4 procedures until and including closure of the wound. The wound closed an average of 7 days (range, 5-14) after placement of the NPWT device. At minimum follow-up of one year, all but 2 achieved a clean, closed wound without removal of instrumentation. Only culture of methicillin-resistant Staphylococcus aureus or multiple bacteria appeared to be related to repeat NPWT procedures. The authors conclude that NPWT “may be an effective adjunct in closing spinal wounds even after the repeat procedures.” Data from case series must be interpreted with great caution.

A number of reports of NPWT in the management of sternotomy wounds were identified in the search, most small case series from outside the US and some that included little data with respect to clinical outcomes including time to healing. Tocco and colleagues report on 21 patients with mediastinitis after sternotomy all of whom were treated with NPWT for an average of 26 days (range, 14-37). (24) Once wound tissue appeared viable and cultures were negative, the chest was closed; in 9 cases using pectoralis flaps, in 9 using Nitinol clips, in one with a combination of muscle flap and Nitinol clips, and in one with sternal wires. Canadian researchers studied predictors of failure of NPWT closure of sternotomy wounds. (25) Twelve risk factors for impaired wound healing were identified before data collection to retrospectively evaluate predictors of NPWT failure. Of 37 patients treated with NPWT between January 1997 and July 2003, 8 patients failed NPWT. Of the 12 risk factors, 3 were found to be predictive of poor outcome: bacteremia, wound depth of 4 or more cm, and high degree of bony exposure and sternal instability. The authors advise that prospective randomized studies are needed to validate these hypotheses.

Svensson and colleagues retrospectively reviewed records of 33 patients who had NPWT of perivascular surgical site infections in the groin after arterial surgery between August 2004 and December 2006. Median duration of NPWT was 20 days and 27 wounds (82%) healed within 55 days. (26) One serious NPWT-associated bleeding and 3 late false femoral artery aneurysms were reported. Synthetic vascular graft infection (n=20) was associated with adverse infection-related events.

An article that systematically reviewed the evidence was published in 2008 by Gregor and colleagues. (27) The authors noted that although there is some indication that NPWT may improve wound healing, the body of evidence is insufficient to clearly prove and additional clinical benefit of NPWT. They also noted that the large number of prematurely terminated and unpublished trials is reason for concern.

An Agency for Healthcare Research and Quality (AHRQ) technology assessment was performed for the Centers for Medicare and Medicaid Services (CMS) and is posted on the AHRQ website. (Negative Pressure Wound Therapy Devices. Technology Assessment Report. Project ID: WNDT1108. May 26, 2009. [28])

This technology assessment of negative pressure wound therapy devices was looking primarily for “therapeutic distinctions” between the various negative pressure wound therapy devices on the market. The Medicare Improvements for Patient and Providers Act (MIPPA) of 2008 called for an evaluation of the HCPCS coding decisions for these devices so this assessment was performed to inform that evaluation. The AHRQ assessment was performed by ECRI Institute and found that there were no studies showing a therapeutic distinction between these devices.

Excerpts from the summary are noted below:

We identified a total of 23 other systematic reviews, all published between 2000 and 2008, that covered NPWT devices. These reviews included studies reporting data on NPWT for patients with a broad range of wound types and focused on comparison to other wound treatments (gauze, bolster dressings, wound gels, alginates, and other topical therapies). The systematic reviews of NPWT reveal several important points about the current state of the evidence on this technology. First, all of the systematic reviews noted the lack of high-quality clinical evidence supporting the advantages of NPWT compared to other wound treatments. The lack of high-quality NPWT evidence resulted in many systematic reviewers relying on low-quality retrospective studies to judge the efficacy of this technology. Second, no studies directly comparing different NPWT components (such as foam vs. gauze dressings) were identified by any of the reviewers.

The authors of this report also comment on a study by Peinemann and colleagues (29) as follows:

In their systematic review of clinical studies of NPWT, Peinemann et al. sought to identify unpublished completed or discontinued RCTs to gain a broader knowledge of the NPWT evidence. The authors were concerned that previous systematic review conclusions on efficacy and safety based on published data alone may no longer hold after consideration of unpublished data. The authors invited two NPWT device manufacturers KCI. (V.A.C.®) and BlueSky Medical Group Inc. (Versatile 1 Wound Vacuum System) and authors of conference abstracts to provide information on study status and publication status of sponsored trials. Responses were received from 10 of 17 (59%) authors and both manufacturers. BlueSky Medical Group Inc., however, had not sponsored relevant [randomized, controlled trials] and only provided case reports. The authors determined that of 28 [randomized, controlled trials], 13 had been completed, six had been discontinued, six were ongoing, and the status of three could not be determined. Nine trials were unpublished, and no results were provided by the investigators. Peinemann et al. concluded that the ‘lack of access to unpublished study results data raises doubts about the completeness of the evidence base on NPWT.”

Chio and Agarwal recently published results of a randomized trial of 54 patients comparing a negative pressure dressing with a static pressure dressing. (30) There were no statistically significant differences in wound complications or graft failure (percentage of area for graft failure was 7.2% for negative pressure and 4.5% for standard dressing). The authors concluded that negative pressure dressing does not appear to offer a significant improvement over standard pressure dressing in healing of the radial forearm free flap donor site.

2011 Update

A search of the MEDLINE database from January 2010 through February 2011 found a systematic review as well as case series, retrospective comparative studies, and concurrent comparative studies (generally not randomized); most studies were from non-US centers. Xie and colleagues identified 17 randomized controlled trials (RCTs) of NPWT that met their criteria for systematic review and found consistent evidence of benefit compared with control treatments for diabetic foot ulcers and conflicting evidence for pressure ulcers. (31) In trials involving mixed wounds, evidence was encouraging but the trials were of inadequate quality. An expert panel convened to develop evidence-based recommendations for the use of NPWT reported that the present evidence base is strongest for the use of NPWT on skin grafts and weakest as a primary treatment for burns. (32) An analysis of NPWT for patients with infected sternal wounds concluded based on 6 articles and 321 patients that NPWT resulted in a decrease of 7.2 days in hospital length of stay with no significant impact on mortality. (33)

Two recent papers report identifying groups of patients who may not benefit from NPWT. Schmelzle et al. reviewed records of 49 patients with open abdomen for more than 7 days due to secondary peritonitis who underwent NPWT. (34) Fascial closure could be accomplished in only 11 patients and complications occurred in 43 patients. Re-explorations after starting NPWT were associated with the occurrence of enterocutaneous fistula and were of prognostic value regarding the rate of fascial closure. The authors advise that further studies are needed to evaluate whether this subgroup really benefits from NPWT. A retrospective multicenter study of hospitalized patients with spinal cord injuries and stage III/IV pelvic pressure ulcers treated with standard wound care (n=53) or NPWT (n=33) measured wound surface over a 28-day observation period. (35) Over the 28-day period, 59 patients' wounds were classified as healing and 27 as nonhealing. The proportion of patients demonstrating a decrease in wound surface area (healing subgroup) was not significantly different between the NPWT and standard care groups. Over the 28-day period, serum albumin concentrations were significantly improved in the healing groups whether or not treated with NPWT. The authors noted that “NPWT may have partially contributed to the lower (or to maintaining the lower) serum albumin concentrations in persons who have malnutrition and a reduced ability to compensate for the wound-related protein loss”.

The FDA has not cleared any NPWT devices for use in children, however a number of case reports and very small case series report experience with infants and small children most commonly for treatment of sternal wounds. (36) A U.S. center reported using gauze based NPWT after skin grafting in an infant burn patient. (37)

Non-Powered Devices

One ultraportable, non-powered (mechanical) gauze based NPWT device (SNaP Wound Care System) designed to remove small amounts of exudate from chronic, traumatic, dehisced, acute, subacute wounds and diabetic and pressure ulcers became available in 2009.

Armstrong and others report results of a planned interim analysis of a randomized controlled trial comparing SNaP and the KCI Wound VAC Therapy System for the treatment of chronic lower extremity wounds. (38) Patients had venous or diabetic ulcers with surface area between 1 and 100 cm2 and diameter less than 10 cm and present more than 30 days despite appropriate care. Dressings were changed per manufacturer direction, 2 times per week in the SNaP group and 3 times per week in the VAC group. Analysis after 65 patients had enrolled was based on 53 patients who had completed at least 4 weeks of therapy, 27 SNaP and 26 VAC. This analysis found no significant between group differences in the proportion of subjects healed or the percent of wound size reduction (p<0.05). Survey data indicated that dressing changes required less time and use of the SNaP device interfered less with mobility and activity than the VAC device. At the start of treatment, wounds in the VAC group were larger (VAC 8.8±9.7 cm2 vs. SNaP 4.3±4.1 cm2) and of longer duration (13.7 months vs. 8.3 months than wounds in the SNaP group). This study does not provide comparison with standard treatment protocols. The authors emphasize that the wounds treated in this study were predominantly secondary to venous disease and therefore were generally more superficial.

A retrospective study with historical controls compared NPWT using the SNaP device (n=28) with wound care protocols that included the use of Apligraf, Regranex, and skin grafting (n=42) for treatment of lower extremity ulcers. (39) Seven patients (25%) in the SNaP treated group could not tolerate the treatment and were discontinued from the study because of complications (allergic skin reaction [1], wound infection [1], bleeding after debridement preventing reapplication [1], worsening lower extremity edema [1], and the development of maceration severe enough to required discontinuation [n=3]) and were considered treatment failures. Eighteen of the remaining 21 patients treated with the SNaP device demonstrated a statistically significant healing trend (p<0.05). Between-group estimates of time to wound healing by Kaplan-Meier analysis were statistically significantly in favor of the SNaP treatment group. Multiple modalities were used in treatment of historical controls. The authors acknowledge that the large number of dropouts and the limitations of retrospectively controlled studies and note that patients in the SNaP treated group may have benefited from being in an experimental environment particularly because wounds in this group were seen twice per week compared to variable follow-up in the historical controls possibly resulting in more frequent debridement in the experimental group.

Other publications have described use of the SNaP device in case series with small numbers of patients, fewer than 15 patients. (40, 41, 42) Landsman comments that by removing compliance barriers this device may encourage more frequent use of NPWT for small wounds. (42)

Medicare Policy

In October 2000, Healthcare Financing Administration (HCFA; now Centers for Medicare and Medicaid Services, CMS) issued the following DMERC (durable medical equipment regional carrier) coverage policy, which stated that patients meeting the following criteria would be eligible for negative wound pressure therapy in the home setting:

Patient has a chronic Stage III or Stage 1V pressure ulcer, venous or arterial insufficiency ulcer, or a chronic ulcer of mixed etiology. A complete wound therapy program should have been tried or considered and ruled out prior to application of negative pressure wound therapy.

### REFERENCES

1. Blue Cross and Blue Shield Association Technology Evaluation Center (TEC). Vacuum-assisted closure in the treatment of chronic wounds. TEC Assessments 2000; Volume 15, Tab 23
2. Ford C N, Reinhard E R, Yeh D e t al. Interim analysis of a prospective, randomized trial of vacuum-assisted closure versus the HealthPoint system in the management of pressure ulcers. Ann Plast Surg 2002; 49(1):55-61.
3. Wanner M B, Schwarzl F, Strub B et al. Vacuum-assisted wound closure for cheaper and more comfortable healing of pressure sores: a prospective study. Scand J Plast Reconstr Surg Hand Surg 2003; 37(1):28-33.
4. Doss M, Martens S, Wood J P et al. Vacuum-assisted suction drainage versus conventional treatment in the management of poststernotomy osteomyelitis. Eur J Cardiothorac Surg 2002; 22(6):934-8.
5. Eginton M T, Brown K R, Seabrook G R et al. A prospective randomized evaluation of negative-pressure wound dressings for diabetic foot wounds. Ann Vasc Surg 2003; 17(6): 645-9.
6. Moues C M, Vos M C, van den Bemd G J et al. Bacterial load in relation to vacuum-assisted closure wound therapy: a prospective randomized trial. Wound Repair Regen 2004; 12(1): 11-7.
7. Samson D J, Lefevre F, Aronson N. Wound-healing technologies: low-level laser and vacuum-assisted closure. Evidence Report/Technology Assessment No. 111. (Prepared by the Blue Cross and Blue Shield Association Technology Evaluation Center Evidence-based Practice Center, under Contract No. 290-02-0026.) A HR Q Publication No. 05-E005-2. Rockville, Md.: Agency for Healthcare Research and Quality. December 2004.
8. Armstrong D G, Layery L A, for the Diabetic Foot Study Consortium. Negative pressure wound therapy after partial diabetic foot amputation: a multicentre, randomized controlled trial. Lancet 2005; 366(9498):1704-10.
9. Armstrong D G, Layery L A, Boulton A J. Negative pressure wound therapy via vacuum-assisted closure following partial foot amputation: what is the role of wound chronicity? Int Wound J 2007; 4(1):79-86.
10. Braakenburg A, Obdeijn M C, Feitz R et al. The clinical efficacy and cost effectiveness of the vacuum-assisted closure technique in the management of acute and chronic wounds: a randomized controlled trial. Plast Reconstr Surg 2006; 118(2):390-7.
11. Vuerstaek J D, Vainas T, Wuite J et al. State-of-the-art treatment of chronic leg ulcers: a randomized controlled trial comparing vacuum-assisted closure (V.A.C.) with modern wound dressings. J Vase Surg 2006; 44(5):1029-38.
12. Ubbink D T, Westerbos S J, Evans D et al. Topical negative pressure for treating chronic wounds. Cochrane Database Syst Rev 2008; (3):C D001898. Update of: Cochrane Database Syst Rev. 2001; (1):C D001898.
13. Wasiak J, Cleland H. Topical negative pressure (T NP) for partial thickness burns. Cochrane Database Syst Rev 2007; (3):C D006215.
14. Gregor S, Maegele M, Sauerland S et al. Negative pressure wound therapy: a vacuum of evidence? Arch Surg 2009; 143(2):189-96.
15. Vikatmaa P, Juutilainen V, Kuukasjarvi P et al. Negative pressure wound therapy: a systematic review on effectiveness and safety. Eur J Vasc Endovasc Surg 2008; 36(4): 438-48.
16. Noble-Bell G, Forbes A. A systematic review of the effectiveness of negative pressure wound therapy in the management of diabetes foot ulcers. Int Wound J 2008; 5(2):233-42.
17. Mody G N, Nirmal I A, Duraisamy S et al. A blinded, prospective, randomized controlled trial of topical negative pressure wound closure in India. Ostomy Wound Manage 2008; 54 (12):36-46.
18. Stannard J P, Robinson J T, Anderson E R et al. Negative pressure wound therapy to treat hematomas and surgical incisions following high energy trauma. J Trauma 2006; 60(6): 1301-6.
19. Shilt J S, Yoder J S, Manuck T A et al. Role of vacuum-assisted closure in the treatment of pediatric lawnmower injuries. J Pediatr Orthop 2004; 24(5):482-7.
20. Yang C C, Chang D S, Webb L X. Vacuum-assisted closure for fasciotomy wounds following compartment syndrome of the let. J Surg Orthop Adv 2006; 15(1):19-23.
21. Zannis J, Angobaldo J, Marks M et al. Comparison of fasciotomy wound closures using traditional dressing changes and the vacuum-assisted closure device Ann Plast Surg 2009; 62(4):407-9.
22. Van Rhee M A, de Klerk L W, Verhaar J A. Vacuum-assisted wound closure of deep infections after instrumented spinal fusion in six children with neuromuscular scoliosis. Spine J 2007; 7(5)596-600.
23. Ploumis A, Mehbod A A, Dressel T D et al. Therapy of spinal wound infections using vacuum-assisted wound closure: risk factors leading to resistance to treatment. J Spinal Disord Tech 2008; 21(5):320-3.
24. Tocco M P, Costantino A, Ballardini M et al. Improved results of the vacuum assisted closure and Nitinol clips sternal closure after postoperative deep sternal wound infection. Eur J Cardiothorac Surg 2009; 35(5):833-8.
25. Gdalevitch P, Afilalo J, Lee C. Predictors of vacuum-assisted closure failure of sternotomy wounds. J Plast Reconstr Aesthet Surg 2010; 63(1):180-3.
26. Svensson S, Monsen C, Kolbel T et al. Predictors for outcome after vacuum assisted closure therapy of peri-vascular surgical site infections in the groin. Eur J Vasc Endovasc Surg 2008; 36(1):84-9.
27. Gregor S, Maegele M, Sauerland S et al. Negative pressure wound therapy: a vacuum of evidence? Arch Surg 2008; 143(2):189-96.
28. Sullivan N, Snyder D L, Tipton K et al. Technology assessment: Negative pressure wound therapy devices. Available online at www.ahrq.gov/clinic/ta/negpresswtd. Last accessed May 2010.
29. Peinemann F, McGauran N, Sauerland S et al. Negative pressure wound therapy: Potential publication bias caused by lack of access to unpublished study results data. BMC Med Res Methodol 2008; 8:4.
30. Chio E G, Agrawal A. A randomized, prospective, controlled study of forearm donor site healing when using a vacuum dressing. Otolaryngol Head Neck Surg 2010; 142(2):174-8.
31. Xie X, McGregor M, Dendukuri N. The clinical effectiveness of negative pressure wound therapy: a systematic review. J Wound Care 2010; 19(11):490-5.
32. Runkel N, Krug E, Berg L et al. Evidence-based recommendations for the use of negative pressure wound therapy in traumatic wounds and reconstructive surgery: Steps towards an international consensus. Injury 2011; 42(Suppl 1):S1-12.
33. Damiani G, Pinnarelli 1, Sommella L et al. Vacuum-assisted closure therapy for patients with infected sternal wounds: a meta-analysis of current evidence. J Plast Reconstr Aesthet Surg 2011 Jan. 21 [Epub ahead of print].
34. Schmelzle M, Alldinger I, Matthaei H et al. Long-term vacuum-assisted closure in open abdomen due to secondary peritonitis: a retrospective evaluation of a selected group of patients. Dig Surg 2010; 27(4):272-8.
35. Ho C H, Powell H L, Collins J F et al. Poor nutrition is a relative contraindication to negative pressure wound therapy for pressure ulcers: preliminary observations in patients with spinal cord injury. Adv Skin Wound Care 2010; 23(11):508-16.
36. Ugaki S, Kasahara S, Arai S et al. Combination of continuous irrigation and vacuum-assisted closure is effective for mediastinitis after cardiac surgery in small children. Interact Cardiovasc Thorac Surg 2010; 11(3):247-51.
37. Psoinos C M, Ignotz R A, Lalikos J F et al. Use of gauze-based negative pressure wound therapy in a pediatric burn patient. J Pediatr Surg 2009; 44(12):e23-6.
38. Armstrong D G, Marston W A, Reyzelman A M et al. Comparison of negative pressure wound therapy with an ultraportable mechanically powered device vs. traditional electrically powered device for the treatment of chronic lower extremity ulcers: a multicenter randomized controlled trial. Wound Rep Regen 2011; 19(2):173-80.
39. Lerman B, Oldenbrook L, Eichstadt S L et al. Evaluation of chronic wound treatment with the SNaP wound care system versus modern dressing protocols. Plast Reconstr Surg 2010; 126(4):1253-61.
40. Fong K D, Hu D, Eichstadt S L et al. Initial clinical experience using a novel ultraportable negative pressure wound therapy device. Wounds 2010; 22(9):230-6.
41. Lerman B, Oldenbrook L, Ryn J et al. The SNaP Wound Care System: a case series using a novel ultraportable negative pressure wound therapy device for the treatment of diabetic lower extremity wounds. J Diabetes Sci Techno12010; 4(4):825-30.
42. Landsman A. Analysis of the SNaP Wound Care System, a negative pressure wound device for treatment of diabetic lower extremity wounds. J Diabetes Sci Technol 2010; 4(4):831-2. Information available in: https://www.bcidaho.com/providers/medical_policies/dme/m_{—}0116.asp

### BRIEF SUMMARY OF THE INVENTION

The dressing is handcrafted using polyurethane or polivynil-alcohol foam. Manual pump (Jackson Pratt or Portovac type, specifically) tube is inserted into the foam and the components are placed on the wound over a of non-adherent gauze. An outer auto-adhesive plastic sheet layer prevents air leakage after activating negative pressure using the manual pump. The system was designed to be cost-effective for widespread use in a out-patient regimen. The dressing should be provided in a single ready-to-use kit that must be designed exclusively to treat open wounds of various origins with the system above stated.

### BRIEF DESCRIPTION OF THE SEVERAL VIEWS OF THE DRAWING

FIG. 1. Scheme of the dressing. By means of the manual pump, negative pressure is transmitted through the siliconized tube into the wound, that is sealed with the outer plastic sheet layer. The effect is faster contraction of the wound, providing healing without the need of further surgical procedures. This device is cheaper than options provided today and should be provided as a ready-to-use-kit accompanied with an waist bag to be distributed with portovac or Jackson-Pratt drains looking forward widespread use.

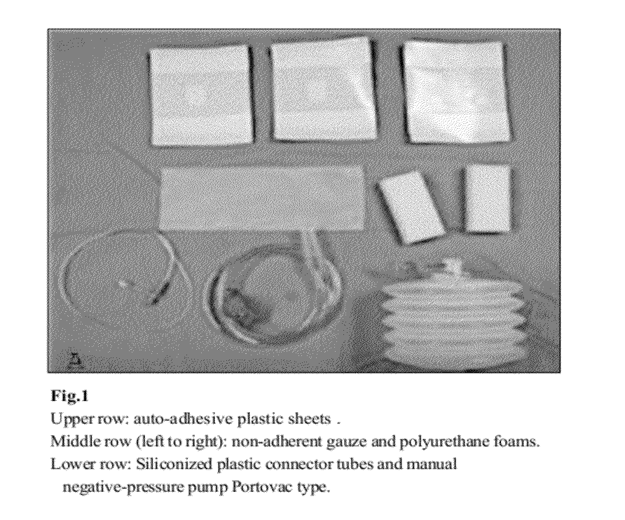

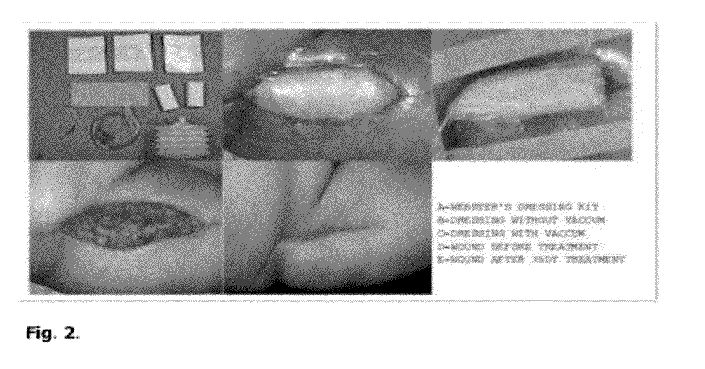

### DETAILED DESCRIPTION OF THE INVENTION

A ready-to-use kit that contains all the needed parts to have the dressing done looking forward simplicity, agility and low costs. The dressing is handcrafted using polyurethane or polivynil-alcohol foam. Manual pump (Jackson Pratt or Portovac type, specifically) tube is inserted into the foam and the components are placed on the wound over a of non-adherent gauze. An outer auto-adhesive plastic sheet layer prevents air leakage after activating negative pressure using the manual pump.

The kit should provide parts (foams, non-adherent gauzes, auoto-adhesive plastic or siliconized sheets and tubes) in diverse dimensions and forms, according the nature of the wound, adapters to the most common devices already in use to provide the vacuum source. The foam design must include within it a channel of proper dimensions to adapt into it the tube originated from the vacuum source. The foam should be made prefereably with open cell technology. The outer plastic adhesive sheet must have extra stregth to prevent air leaks specificallly for the presented purpose of treat open wounds. The passage of the tube through the plastic sheet must be done in a specific manner where the tube must not get in contact with the healthy skin. The tube must be totally wrapped with the auto-adhesive plastic sheet and only the plastic sheet must adhere to the skin. This must be stressed in the directions of use of the dressing. The kit must contain a waist bag to provide easy portability of the manual pump.

1: The isolated elements needed in the dressing should be offered as a ready-to-use kit that will allow a easy way to deal with complex open wounds. This action will allow a widespread use of a cost-effective vacuum dressing, as above stated, mainly in the out-patient regimen. The main purpose of this kit is to be distributed with or as a complimentary part of Jackson-Pratt or Portovac style suction drains, specifically.
a: The kit must provide parts in diverse dimensions and forms, according the nature of the wound.
b: The kit must contain adapters to the most common devices already in use to provide the vacuum source.
c: the foam design must include within it a channel of proper dimensions to adapt into it the tube originated from the vacuum source. The foam should be made prefereably with open cell technology.
d: The outer plastic adhesive sheet must have extra stregth to prevent air leaks specificallly for the presented purpose of treat open wounds.
e: The kit must contain a waist bag to provide easy portability of the manual pump.
